(19) United States
(12) Patent Application Publication
(10) Pub. No.: US 2026/0315550 A1
(43) Pub. Date: Oct. 15, 2026

(54) BACTERIA STRAINS FOR THE TREATMENT OF UROLOGICAL DISORDERS, THEIR COMPOSITIONS AND USES THEREOF

(71) Applicant: PROBIONOVA SA, Lugano (CH)

(72) Inventors: Marco BOCCARUSSO, Lugano (CH); Giorgio Stefano CERANA, Lugano (CH); Peter FRANCK, Lugano (CH)

(21) Appl. No.: 19/119,086

(22) PCT Filed: Oct. 31, 2023
(86) PCT No.: PCT/IB2023/060965

§ 371 Date: Apr. 7, 2025

(30) Foreign Application Priority Data
Oct. 31, 2022 (IT) 102022000022401

(51) Int. Cl. A61K 35/745; A61P 13/08

(52) U.S. Cl. CPC A61K 35/745; A61P 13/08

(57) ABSTRACT

The present invention relates to selected bacterial strains belonging to the Lactobacillus and Bifidobacterium species and their mixtures, compositions, and use in therapy, especially in the male urological field for the treatment of male fertility. More specifically, the invention relates to the use of specific bacterial strains in the curative or prophylactic treatment of benign prostatic hyperplasia and myometrial dysfunction. In addition, the present invention relates to selected bacterial strains, belonging to the species Lactobacillus and Bifidobacterium, and mixtures and compositions thereof, for use in a method of treatment, preferably a preventive or curative method of treatment, of male urinary diseases, preferably of benign prostatic hyperplasia (BPH). Finally, the present invention relates to selected bacterial strains, belonging to the species Lactobacillus and Bifidobacterium, and mixtures and compositions thereof, for use in a method of treatment, preferably a preventive or curative method of treatment, of gynecological diseases, preferably of diseases associated with alterations in myometrial trophism and endocrine and metabolic disorders such as polycystic ovary syndrome (PCOS), endometrial hyperplasia, and endometriosis.

The present invention relates to selected bacterial strains belonging to the Lactobacillus and Bifidobacterium species and their mixtures, compositions, and use in therapy, especially in the male urological field for the treatment of male fertility. More specifically, the invention relates to the use of specific bacterial strains in the curative or prophylactic treatment of benign prostatic hyperplasia and myometrial dysfunction. In addition, the present invention relates to selected bacterial strains, belonging to the species Lactobacillus and Bifidobacterium, and mixtures and compositions thereof, for use in a method of treatment, preferably a preventive or curative method of treatment, of male urinary diseases, preferably of benign prostatic hyperplasia (BPH). Finally, the present invention relates to selected bacterial strains, belonging to the species Lactobacillus and Bifidobacterium, and mixtures and compositions thereof, for use in a method of treatment, preferably a preventive or curative method of treatment, of gynecological diseases, preferably of diseases associated with alterations in myometrial trophism and endocrine and metabolic disorders such as polycystic ovary syndrome (PCOS), endometrial hyperplasia, and endometriosis.

### Urological Disorders

One of the most frequently diagnosed urologic diseases in men over the age of 50 is benign prostatic hyperplasia (BPH).

Among the most significant manifestations of BPH are lower urinary tract symptoms (LUTS) and benign prostatic enlargement (BPE); normally LUTS arise due to bladder outlet obstruction (BOO), which leads to benign prostatic obstruction (BPO). The development of BPH is often associated with other preexisting pathologies; in fact, a relationship between metabolic syndrome (MetS) and the risk of LUTS and BPH has already been demonstrated.

In the '50s it was discovered that there is a link between BPH and prostate cancer (PCa): both arise due to chronic inflammation, depend on the presence of androgens, and their development may be associated with metabolic factors.

Considering that short-chain fatty acids (SCFAs) have a very high immunomodulatory potential, more and more research is currently being conducted on their effect not only in the gut microenvironment, but also in cells and tissues of other organs.

Short-chain fatty acids (SCFAs) have less than 6 carbon atoms in their chain (C1-C6); the most common SCFAs generated by particular gut microbiomes are acetate (C2), propionate (C3), and butyrate (C4). It is known that SCFAs play a role in the regulation of host health or disease mainly through two mechanisms: the regulation of target cell epigenetics after SCFAs enter the cell and signal transduction through membrane receptors; in fact, one of the main features of SCFAs is their activity as endogenous inhibitors of HDACs (HDACi), especially propionate and butyrate.

These acids are present in humans in certain amounts, and concentrations depend on the ratio of bacteria inhabiting the gut and disorders of the gut microflora (dysbiosis), which affects the amount of SCFAs produced. Thus, SCFAs levels vary depending on the composition of the gut microflora and food intake; however, differences in analytical methods and methodology of preparing material for research on the correlation between SCFAs and disorders such as BPH cause difficulties in interpreting the results obtained.

The influence of SCFAs on the development of BPH has not been fully studied. Only a few publications on the impact of gut microflora on the prostate can be found in the literature. They mainly concern the influence of gut bacteria on the synthesis of metabolites and androgens, which may be associated with the development of prostate cancer in humans. There are also reports on the impact of inflammatory bowel disease (IBD) on prostate cancer risk. So far, differences in the composition of gut microflora have been confirmed only in a pilot study, in which the composition of gut microflora in patients with PCa and BPH was analyzed. It has been observed that chronic inflammation of the prostate predisposes to BPH and PCa; this inflammation may be caused by bacterial infection, but may also be associated with low-grade systemic inflammation. It seems most likely, however, that altered gut microflora does not directly affect the prostate gland, but contributes to the development of chronic systemic inflammation. Cells and inflammatory factors (including cytokines) of the gut environment, along with circulation, can enter the gland and cause “local” inflammation and stimulate growth factors in the prostatic stroma, which in turn can lead to prostatic hyperplasia. The influence of gut microflora and its metabolites entering the systemic circulation has been confirmed by studies on the urinary, nervous and respiratory systems, as well as on autoimmune diseases.

In patients with BPH, a stool analysis showed increased levels of some SCFAs, mainly isobutyric acid (C4:0i), isovaleric acid (C5:0i) and isocaproic acid (C6:01); from this it can be inferred that these are the ones most likely to influence the factors that predispose men to BPH. Specifically, isobutyric acid was significantly elevated in men with BPH compared to healthy controls (% SCFA—mean: 4.695, median: 4.702 vs. 3.814, 3.726; p=0.008). Isovaleric acid levels were also higher (% SCFA—mean: 9.499, median: 9.221 vs. 6.911, 6.730; p<0.001). Finally, the acid that predominated among the acids isolated from the feces of healthy controls was isocaproic acid (% SCFAs—mean: 0.359, median: 0.174 vs. 0.186, 0.142; p=0.038). SCFAs produced by the gut microbiota, mainly acetate, propionate and butyrate, play key roles in maintaining homeostasis in humans. These three most common acids account for 95 percent of total SCFAs. In the large intestine and stool samples, SCFAs are present in an approximate molar ratio of acetic acid:propionate:butyric acid of 60:20:20. Levels of SCFAs in the intestine range from 20 to 140 mM and depend on the composition of the intestinal microflora, the absorption of SCFAs from the intestine, and the fiber content of the diet. Acetic, propionic and butyric acids are produced as a result of saccharolytic fermentation and have health benefits.

SCFAs are considered mediators in the communication between the gut microbiome and the immune system. Among other things, the signal they produce is transferred into immune cells via free fatty acid receptors (FFARs), which belong to the G-protein-coupled receptor (GPCR) family. Considering their immunomodulatory potential, they may be useful in preventing chronic but persistent low-grade inflammation. It is also worth noting that SCFAs and BCFAs (branched-chain fatty acids) concentrations fluctuate in a healthy population throughout life, from infants to the elderly. These values are influenced by the composition of the gut microflora, age, and health of the subjects. Many studies have confirmed that the gut microflora changes as the body ages. It has been observed that the positive impact of microflora (characterized by a decrease in the taxonomic diversity of the gut microbiota) on the human body, decreases with age. These changes are also reflected in the physiology of the host organism and manifest as increased inflammation, among other things. These differences can be seen between adult men (average age 42 years) and elderly men (average age 77 years). In young men, the bacteria that predominate in the composition of the intestinal microflora are those with immunomodulatory potential, such as Clostridiales and Bifidobacterium. In the elderly, instead, bacterial communities are enriched with pathogens, e.g., Enterobacteriaceae. Butyric acid (C4:0) is an important factor influencing metabolic processes, which has been confirmed in both animal models and humans, but the exact mechanism of its action still requires more detailed explanation. Butyric acid supplementation has been shown to prevent obesity, hyperinsulinemia, and hypertriglyceridemia and may also reduce appetite and activate brown adipose tissue (BAT) through vagal nerve signaling. Butyric and valeric acids have also been shown to be class I histone deacetylase inhibitors.

Isobutyric acid (C4:0i) is a short-chain branched fatty acid (BCFA), a geometric isomer of butyric acid, which results in its different physical but not chemical properties. In the human intestine, fermentation of branched-chain amino acids is carried out mainly by the genera Bacteroides and Clostridium. Among the bacterial populations involved in peptide and amino acid fermentation are those bacteria responsible for BCFAs production: 0.6% of the population is involved in isovaleric acid synthesis and up to 40% of the bacteria in isobutyric acid synthesis. The highest levels of BCFAs are found in the distal parts of the large intestine. BCFAs concentrations in feces, as well as SCFAs concentrations, may be altered depending on the food consumed. So far, little is known about the effects of BCFAs on the host organism. However, there is evidence that these acids can be oxidized if the amount of butyric acid is insufficient and thus can be a source of energy for colonocytes. It has been shown that isobutyric acid (C4:0i), isovaleric acid (C5:0i), and isocaproic acid (C6:0i) are the ones most likely influencing the factors that predispose humans to BPH. In addition, during proteolytic fermentation, along with the increase in BCFAs, harmful metabolites such as: ammonia, phenols, and hydrogen sulfide are produced. The resulting components may contribute to the initiation of inflammation and excessive colonocyte proliferation and thus influence local disease states. In addition to inducing epithelial cell proliferation, inflammation can also affect tight junctions (TJs). Damage to tight junctions causes loss and dysfunction of cellular barriers and is the cause of “leaky states” and chronic inflammation, which are observed in digestive tract cancers. Impairment of tight junction function in the digestive tract (mainly in the intestine) can also be caused by the state of intestinal dysbiosis and the reduced amount of SCFAs, resulting from, among other things, the use of antibiotics. SCFAs, mainly butyric acid, strengthen the intestinal barrier by regulating the transcription of claudin-1, which is a protein that builds tight junctions. Leakage of the intestinal barrier causes bacterial particles and factors, pro-inflammatory cytokines, immune cells, toxins and antigens to enter the bloodstream and move from the digestive tract to distant places, including the prostate. Many studies have confirmed that SCFAs are involved in the pathophysiology of IBD and may be a prognostic marker of disease status.

Research has also confirmed the role of SCFAs in the pathogenesis of IBD. Their analysis showed that in people with inflammatory bowel disease, the levels of the main fatty acids—acetic, butyric, and propionic—decrease dramatically compared with healthy people (162.0; 86.9; 65.6 μmol/g vs. 209.7; 176.0; 93.3 μmol/g). In addition, the effect of L. plantarum in alleviating the symptoms of IBS with predominant diarrhea (IBS-D) was investigated: this study showed how L. plantarum significantly restores the composition and diversity of the intestinal microbiota, in particular its benefits may be related to the increase of bacteria capable of producing butyric acid.

Changes in fecal short-chain fatty acids were also observed in patients with pathological obesity after surgery. When they lost weight and changed their diet, the total amount of SCFAs decreased. A similar effect was obtained for the relative amounts of linear-chain SCFAs, namely acetic, propionic and butyric acids. At the same time, the levels of BCFAs (isobutyric, isovaleric and isocaproic acids) increased. These changes suggest a predominant proteolytic fermentation that may have adverse health effects. One therapy may be to change a protein-rich diet to one rich in carbohydrates, fiber, and polysaccharides in order to increase saccharolytic fermentation and the level of the major simple SCFAs (acetate, propionate, and butyrate), with health-beneficial properties.

Butyrate levels are also altered in prostate cancer patients; in one study it was observed that several pathways leading to butyrate production are activated in greater abundance than in benign controls. In addition, sodium butyrate (the sodium salt of butyric acid) has been shown to decrease androgen receptor expression in some prostate cancer cell lines.

Herbal remedies, pharmacotherapy, and surgery are currently available for BPH. European and non-European guidelines focusing on therapeutic options for the disease also include pharmacotherapy, lifestyle recommendations, surgical options, and phytotherapy. In addition, researchers are focusing on new herbal alternatives regarding the treatment of BPH, particularly traditional Chinese medicine.

Drugs based on the pharmacological actions of active compounds include.
α1-adrenergic antagonists
5α-reductase inhibitors
muscarinic receptor antagonists
Type 5 phosphodiesterase inhibitors and vasopressin analogs.

### Gynecological Disorders.

Currently, there is ample evidence showing that many of the bacteria that colonize the human body have established beneficial relationships with their host, actively participating in the formation and maintenance of normal physiological balance. In contrast, dysbiosis promotes the development of various diseases. In fact, microbial colonies living in the human body play an important role in the state of eubiosis of any individual; in particular, the cervicovaginal microbiome plays a key role in women's reproductive health by interacting with estrogen, androgen, insulin, and other hormones. Alteration of the natural state of the microbiota has been shown to contribute to endocrine and metabolic disorders such as polycystic ovary syndrome (PCOS), endometrial hyperplasia and endometriosis, and various comorbidities such as diabetes. Unfortunately, data available in the scientific literature are scarce, especially regarding mechanisms.

The gut microbiome modulates many biological mechanisms; for example, commensal bacteria can produce and secrete hormones, and communication between microbes and hormones can influence metabolism, immunity, and host behavior. In addition, sex hormones, such as progesterone, estradiol, and testosterone, also participate in communication between microorganisms and their hosts and play several important physiological roles in cell reproduction, differentiation, proliferation, apoptosis, inflammation, metabolism, homeostasis, and brain function. In this context, several studies have shown that disruption of the vaginal microbiome of women of reproductive age affects all stages of women's reproductive life. More specifically, the human microbiome influences every stage and level of female reproduction, including follicle and oocyte maturation in the ovary, fertilization and migration of the embryo, implantation, and the entire pregnancy, including during childbirth. Alterations in the microbiome, particularly in the gut, have a specific impact on the reproductive endocrine system, and correction of abnormal microbiomes can lead to better reproductive outcomes.

The Human Microbiome Project has shown that the microbiome of a normal vagina is populated by several species of indigenous lactobacilli and that, compared with other body sites, microbial diversity within the reproductive tract is relatively low. In this context, many studies have shown that dietary supplementation or direct intervention with probiotics can alleviate reproductive dysfunction and have positive therapeutic effects on associated diseases; moreover, there are several evidences that probiotics can alter the biological activity of microbes, thus exerting their effects by directly regulating the composition of the host microbiota and also influencing its metabolism and health. In particular, probiotics can improve the reproductive function of the host by regulating its metabolism, since metabolic health is linked to reproductive function.
Document D1 (WO2022/234511A2) filed on 5 May 2022 describes a mixture of bacteria comprising at least one bacterium selected from four strains, including a strain of Bifidobacterium psychraerophilum “Q5.” The mixture is useful for the treatment of alterations in carbohydrate metabolism.
Document D2 (Strains Portfolio—Probionova SA″, 27.9.2022) mentions the species Bifidobacterium psychraerophilum and Bifidobacterium longum.
Document D3 (novaPROX Combo—Probionova SA″, 14.3.2023) is a publication by the same Applicant at a later date.
Document D4 (Boccarusso: “Fermented foods: a traditional source of new probiotics” AGRO FOOD INDUSTRY HI-TECH, Jan. 1, 2021) describes a study conducted on three strains of probiotics including a strain of Bifidobacterium psychraerophilum and their activity at the gut level.
Document D5 (Stefan Siebrecht et al.: “THE INFLUENCE OF PARTIALLY HYDROLYZED GUAR GUM (PHGG) FIBER ON THE INTESTINAL MICROBIOTA,” Jan. 1, 2021) is a publication on the effects of partially hydrolyzed guar gum on the intestinal microbiota.
Document D6 (U.S. Pat. No. 9,855,302B2) describes the treatment of cancer by administering an immune checkpoint inhibitor and a Bifidobacteria-based formulation.
Document D7 (US2008/274085A1) describes a Bifidobacterium that has a DNA sequence more than 40% homologous with Bifidobacterium GC56.
Document D8 (US2022/218761A1) describes a composition of bacteria that includes some strains of Bifidobacterium longum.
Document D9 (U.S. Pat. No. 10,286,017B2) describes the treatment of intestinal disorders in pediatric subjects that comprises administering a mixture of probiotics.
Document D10 (CN110859871A) describes a composition based on mulberry leaves and Bifidobacteria.

In view of the above, it is evident that the identification of new probiotic-based treatments for the urological and gynecological disorders described above as an alternative to conventional phytotherapeutic and pharmacological treatments would be useful.

The Applicant, following intensive and prolonged research and development, has surprisingly found that specific bacterial strains of the genus Bifidobacterium are able to exert important beneficial activity in male urological disorders.

The Applicant also found that specific bacterial strains of the genus Bifidobacterium and Lactobacillus show interesting and unexpected activities at the gynecological level.

In particular, the Applicant surprisingly found that specific bacterial strains of the genus Bifidobacterium, specifically strains:
Bifidobacterium psychraerophilum “Q5”, deposited at the Deutsche Sammlung von Mikroorganismen und Zellkulturen GmbH (DSMZ) under deposit number DSM 33131, by Probionova SA on Jul. 2, 2019, and
Bifidobacterium longum subsp. longum “novaBLG1,” deposited at the Deutsche Sammlung von Mikroorganismen und Zellkulturen GmbH (DSMZ) under deposit number DSM 34338, by Probionova SA on Jul. 26, 2022, and mixtures thereof,
   are able to exert an important beneficial activity towards urological disorders in men particularly, but not only, in the curative or prophylactic treatment of benign prostatic hyperplasia (BPH).

With reference to B. psychraerophilum strain Q5 DSM 33131, the following is reported.

### Growing Medium Recipe:

### MRS-Cysteine Medium:

MRS broth (de Man, Rogosa and Sharpe culture medium) supplemented with cysteine as reducing agent. Ssuitable for anaerobic cultures.

Glucose 20.00 g/l
Peptone 10.00 g/l
Beef extract 8.00 g/l
Sodium acetate trihydrate 5.00 g/l
Yeast extract 4.00 g/l
Dipotassium hydrogen phosphate 2.00 g/l
Triammonium citrate 2.00 g/l
Tween 80 1.00 g/l
Magnesium sulfate heptahydrate 0.20 g/l
Manganese sulfate tetrahydrate 0.05 g/l
L-cysteine HCl monohydrate (*) 0.50 g/l
Final pH 6.2 ± 0.2
(*) L-cysteine HCl should be added after the autoclave cycle.

### Scientific Description:

B. psychraerophilum cells are Gram-positive, catalase- and oxidase-negative, nonmotile, nonspore-forming, short and irregularly shaped, about 0.7-1.0 μm wide and 0.8-1.5 μm long, with occasional bifurcations, arranged singly or in pairs.

Colonies on MRS-cys agar under anaerobic conditions are white, circular and convex with smooth edges and reach a diameter up to 3 mm after a 3-day incubation at 37° C. Colonies also form under aerobic conditions but reach a small diameter of ~1 mm after 3 days of incubation. Optimal temperature for growth is 34-37° C.; maximum temperature for growth is 42° C., with no growth at 46° C. Growth can occur at 4° C., although it is greatly reduced. The lowest pH achieved is 4.0, with a minimum initial pH for growth of 4.5. The G+C content of DNA is 59.2 mol %.

B. psychraerophilum Q5 strain was isolated from water kefir. It appears to be aerotolerant, but preferential growth under anaerobic conditions, and its optimal growth temperature is between 34 and 37° C.

The microbiological and biochemical characteristics reported for B. psychraerophilum type strain LMG 21775 are:

### Characteristics:

Aerobic growth+
Temperature range for growth 4-42° C.
G+C content of DNA: 59.2

### Carbohydrate Profile:

Maltose−
Mannose−
Melezitose+
Xylose+
Salicin+
Arabinose+
Raffinose+
Cellobiose+

In addition, the Applicant surprisingly found that specific bacterial strains of the genus Bifidobacterium and Lactobacillus, particularly the strains:
Bifidobacterium bifidum “novaBBF9” deposited at the Deutsche Sammlung von Mikroorganismen und Zellkulturen GmbH (DSMZ) under deposit number DSM 34337, by Probionova SA on Jul. 26, 2022,
Lactobacillus crispatus “novaLCR” deposited at the Deutsche Sammlung von Mikroorganismen und Zellkulturen GmbH (DSMZ) under deposit number DSM 34348, by Probionova SA on Jul. 26, 2022, and
Lactobacillus fermentum (Limosilactobacillus fermentum) “novaLV58” deposited at the Deutsche Sammlung von Mikroorganismen und Zellkulturen GmbH (DSMZ) under deposit number DSM 34340, by Probionova SA on Jul. 26, 2022, and mixtures thereof,
   are able to exert an important beneficial activity towards gynecological disorders, particularly, but not only, by activating mechanisms impaired under conditions of myometrial disruption and improving myometrial activity through modulation of cell cycle progression.

All the above strains of bacteria have been deposited in accordance with the provisions of the Budapest Treaty; the Depositor of said strains of bacteria described and claimed in this patent application and the Applicant express their consent to make the strains available for the duration of the patent.

### BRIEF DESCRIPTION OF THE FIGURES

FIGS. 1 to 12 relate to the first aspect of the invention, and FIGS. 13 to 21 relate to the second aspect of the invention. In FIGS. 13 to 21, the association A2 is referred to as MIX.

FIG. 1 shows a schematic example of a 3D model of intestinal epithelium connected to the prostate.

FIG. 2 shows the 3D spheroids in culture monitored during growth.

FIG. 3 shows the intestinal passage of metabolites produced by association A1.

FIGS. 4, 5 and 6 show the effects of association A1 on prostate cell activity.

FIG. 7 shows the effects of association A1 on reducing oxidative stress.

FIG. 8 shows the effects of association A1 on reducing inflammation.

FIG. 9 shows the effects of association A1 on androgen receptor (AR) activity.

FIG. 10 shows the effects of association A1 on testosterone levels.

FIG. 11 shows the effects of association A1 on serotonin levels.

FIG. 12 shows the effects of association A1 on prostate-specific antigen (PSA).

FIG. 13 shows the cell viability of PHM1-41 cells incubated with all individual agents for 24 hours as measured by MTT assay; data are the mean±SD of five independent experiments performed in triplicate versus control (0% line). *p<0.05 compared with control; #p<0.05 compared with other concentrations.

FIG. 14 shows mitochondrial metabolism and TEER analysis. In panel A, mitochondrial metabolism assessed by MTT at 6 hours. In B, TEER during 6 hours. Data are expressed as mean±SD (%) of 5 independent experiments normalized to control. *p<0.05 vs control; #p<0.05 vs single agents; !p<0.05 vs Association A2+DCI 150 mg; y p<0.05 vs DCI 150 mg.

FIG. 15 shows the absorption analysis. In panel A, fluorescein rate at 6 hours. In B, quantification of butyric acid measured at 6 hours. Data are expressed as mean±SD (%) of 5 independent experiments normalized to control. *p<0.05 vs control; #p<0.05 vs single agents; !p<0.05 vs Association A2+DCI 150 mg; y p<0.05 vs DCI 150 mg.

FIG. 16 shows the proliferation analysis. In panel A, mitochondrial metabolism assessed by MTT assay. In B, proliferation analysis by crystal violet staining. In C, cyclin D1 activity assessed by ELISA kit. Data are expressed as mean±SD (%) of 5 independent experiments normalized to control. *p<0.05 vs control; #p<0.05 vs single agents; *p<0.05 vs Association A2+DCI 150 mg; y p<0.05 vs DCI 150 mg.

FIG. 17 shows the damage to the myometrium. In panel A, ROS production measured by cytoC reduction. In panel B, TNFa activity analyzed by ELISA kit. Data are expressed as mean±SD (%) of 5 independent experiments normalized to control. *p<0.05 vs control; #p<0.05 vs single agents; !p<0.05 vs Association A2+DCI 150 mg; y p<0.05 vs DCI 150 mg.

FIG. 18 shows the contractile activity. Concentration of oxytocin (A) and MAGT1 activity (B) measured by ELISA kits. Data are expressed as mean±SD (%) of 5 independent experiments normalized to control. *p<0.05 vs control; #p<0.05 vs single agents; !p<0.05 vs Association A2+DCI 150 mg; y p<0.05 vs DCI 150 mg.

FIG. 19 shows the intracellular pathway. Activity of ERK/MAPK (A) and PI3K/AKT (B) assessed by ELISA kits. Data are expressed as mean±SD (%) of 5 independent experiments normalized to control. *p<0.05 vs control; #p<0.05 vs single agents; !p<0.05 vs Association A2+DCI 150 mg; y p<0.05 vs DCI 150 mg.

FIG. 20 shows the intracellular pathways. Activity of ERb (A), PAX8 (B) and PAK1 (C). Data are expressed as mean±SD (%) of 5 independent experiments normalized to control. *p<0.05 vs control; #p<0.05 vs single agents; !p<0.05 vs Association A2+DCI 150 mg; y p<0.05 vs DCI 150 mg.

FIG. 21 shows the hormonal activity. Concentration of LH (A) and FSH (B) measured by ELISA kit. Data are expressed as mean±SD (%) of 5 independent experiments normalized to control. *p<0.05 vs control; #p<0.05 vs single agents; !p<0.05 vs Association A2+DCI 150 mg; y p<0.05 vs DCI 150 mg.

FIG. 22 shows cell viability on CaCo-2 cells incubated with B. longum and B. psychaerophilum at different dosages, for 2 hours (A), 6 hours (B) and 24 hours (C). Data are mean±SD of five independent experiments performed in triplicate versus control values (0% line) and *p<0.05 versus control; **p<0.05 versus single agents; bar p<0.05 versus 1*10⁹ CFU/ml B. longum BLG1+2*10⁹ CFU/ml B. psychraelophilum Q5.

FIG. 23 shows the permeability study on CaCo-2 cells. In (A) the analysis of claudin-1 measured by ELISA assay; in (B) the analysis of ZO-1 by ELISA assay; in (C) the analysis of occludin-1 by ELISA kit;

### DESCRIPTION OF THE INVENTION

According to a first aspect, it is an object of the invention a bacterial strain selected from:
Bifidobacterium psychraerophilum “Q5,” deposited at the Deutsche Sammlung von Mikroorganismen und Zellkulturen GmbH (DSMZ) under deposit number DSM 33131, by Probionova SA on Jul. 2, 2019;
Bifidobacterium longum subsp. longum “novaBLG1,” deposited at the Deutsche Sammlung von Mikroorganismen und Zellkulturen GmbH (DSMZ) under deposit number DSM 34338, by Probionova SA on Jul. 26, 2022;
and mixtures thereof.

It is also an object of the first aspect of the invention a pharmaceutical or nutraceutical or dietary supplement association or combination consisting of the two strains of the invention (association A1) Bifidobacterium psychraerophilum “Q5” DSM 33131 and Bifidobacterium longum subsp. longum “novaBLG1” DSM 34338, preferably in a weight ratio from 1:10 to 10:1, even more preferably in a weight ratio from 1:5 to 5:1, e.g., from 1:3 to 3:1, or from 1:2 to 2:1, or 1:1, preferably all strains of bacteria are in lyophilized form.

It is also an object of the first aspect of the invention a pharmaceutical or nutraceutical or dietary supplement composition comprising only one of the above-described strains or association or combination thereof (A1), optionally together with at least one physiologically and/or pharmaceutically acceptable or food-grade excipient and/or vehicle.

It is also an object of the first aspect of the invention the use of said bacterial strains and/or mixtures thereof in therapy and, more specifically, in the curative or prophylactic treatment of male urinary diseases, preferably benign prostatic hyperplasia (BPH).

It is also an object of the first aspect of the invention the use of the compositions of the invention in therapy and, more particularly, in the curative or prophylactic treatment of male urinary diseases, preferably benign prostatic hyperplasia (BPH).

Preferably, the first aspect of the invention refers to a composition comprising from 1 to 100 mg of B. longum novaBLG1 and from 1 to 100 mg of B. psychaerophilum Q5, both in lyophilized form, preferably from 5 mg to 50 mg of B. longum novaBLG1 and from 5 mg to 50 mg of B. psychaerophilum Q5, even more preferably from 10 mg to 40 mg of B. longum novaBLG1, e.g., 20 mg or 30 mg of B. longum novaBLG1, and from 10 mg to 40 mg of B. psychaerophilum Q5, e.g., 20 mg or 30 mg of B. psychaerophilum Q5, preferably all strains of bacteria are in lyophilized form.

Preferably, a composition may comprise from 5 to 20 mg of B. longum novaBLG1 and from 5 to 20 mg of B. psychaerophilum Q5, both in lyophilized form, preferably 10 mg of B. longum novaBLG1 and 10 mg of B. psychaerophilum Q5, preferably all strains of bacteria are in lyophilized form.

Preferably, the first aspect the invention refers to a composition comprising 10 mg of B. longum novaBLG1 and 20 mg of B. psychaerophilum Q5, both in lyophilized form.

Preferably, compositions of the first aspect of the invention may comprise one or more physiologically and/or pharmacologically acceptable or food-grade excipients and/or vehicles.

Preferably, each of the two strains of the first aspect of the invention is administered in daily doses ranging from 1.0×10⁶ CFU/day to 1.0×10¹² CFU/day, preferably from 1.0×10⁸ CFU/day to 1.0×10¹⁰ CFU/day, e.g., about 1.0×10⁹ CFU/day (CFU=Colony Forming Units).

Compositions can be administered once or several times a day, preferably once a day.

Strains of the first aspect of the invention may be present in the composition of the first aspect of the invention as live and viable bacteria, dead bacteria or cellular components, cell extracts, lysates or tyndallized thereof.

Preferably, compositions of the first aspect of the invention may contain the at least one physiologically and/or pharmacologically acceptable or food-grade excipient having prebiotic characteristics or properties. Preferably, said at least one physiologically and/or pharmacologically acceptable or food-grade excipient may include a monosaccharide, a disaccharide, a polysaccharide, soluble fiber and/or insoluble fiber, GOS, FOS, inulin, maltodextrin, and mixtures thereof.

Preferably, said at least one physiologically and/or pharmacologically acceptable or food-grade excipient may include partially hydrolyzed guar gum (PHGG).

Preferably, said at least one physiologically and/or pharmacologically acceptable or food-grade excipient could be selected from the group comprising or alternatively consisting of: inulin, a fructooligosaccharide (FOS), maltodextrin, and mixtures thereof.

Compositions according to the first aspect of the invention are preferably compositions for oral use, advantageously in capsule form. However, other pharmaceutical forms may be used, such as, for example, powders, tablets, or dispersions.

The two strains of the first aspect of the invention have been the subject of numerous experimental assays, reported below.

During these assays, the Applicant observed that particularly when associated or combined, the probiotic strains of the invention, Q5 and novaBLG1 (association A1), improve androgen receptor function, testosterone level, and serotonin, restoring their balance and improving BPH status (markers evaluated: Ki67, androgen receptor activity, testosterone level, 5Htr1a receptor, serotonin levels, PSA).

All these important effects are believed to be due to the presence of SCFAs secreted by probiotics, e.g., butyric acid, which can act as second messengers and activate various mechanisms impaired in BPH.

In addition, the strains tested did not induce any damage to intestinal epithelia, confirming their nontoxicity and ability to enhance antioxidant mechanisms (markers: ROS, SOD) and their anti-inflammatory activity (markers: TNF-a, IL-6, and IL-10), while also acting on the prostate serotonergic pathway and decreasing proliferative activity.

The assays conducted and the results obtained with the association A1 of the first aspect of the invention will be shown below, with reference to the attached Figures.

### Experimental Section—BPH

### Legend

Longum=B. longum novaBLG1
Psy=B. psychaerophilum Q5
Association=Longum+Psy

### Transwell System

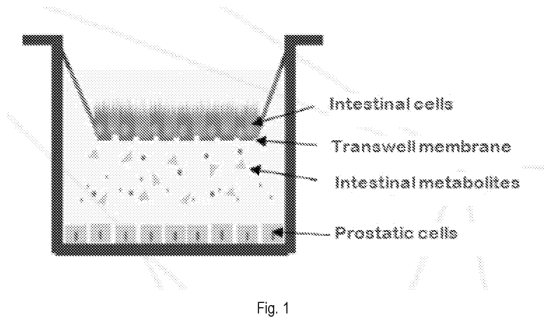

The product metabolized by intestinal cells in a Transwell system was directly contacted with prostate cells seeded in a basolateral environment as reported in the literature, in order to reproduce in vitro what happens in the human body (FIG. 1).

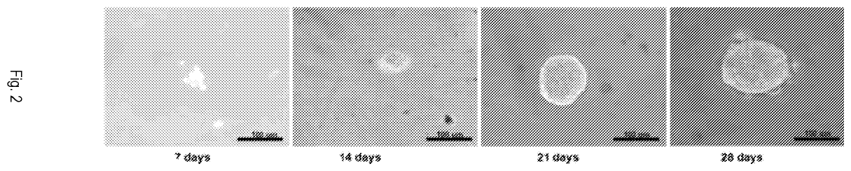

Prostate organoids were pretreated with 0.5 μM testosterone to simulate prostatic hyperplasia in vitro (FIG. 2).

### Intestinal Phase Analysis

These data demonstrate that the selected formulation is able to stimulate the beneficial effect on mitochondrial metabolism after intestinal passage.

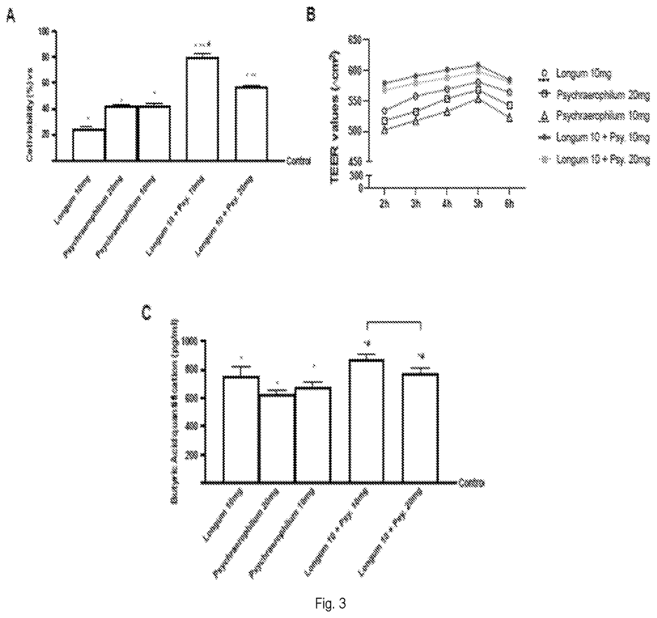

In particular, the association of Longum 10 mg+Psy 10 mg and 20 mg produces no damage in the intestine compared to the single agents, suggesting that these two formulations (FIG. 3A)

Evaluation of transepithelial electrical resistance (TEER) allows the integrity of the cell monolayer to be assessed. TEER values of 500±52.9 Ω*cm 2 for intestinal CaCo 2 cells are in agreement with data available in the literature and suggest that the cells maintain an intact monolayer after treatments (FIG. 3B).

Intestinal passage analysis showed that all selected agents are able to produce butyric acid (probiotic metabolite) as a second messenger. The association increases the intestinal passage of metabolites suggesting that the probiotic formulation is able to directly reach the target organ (FIG. 3C).

### Prostatic Analysis After Intestinal Metabolism

### Assessment of Prostatic Cell Activity

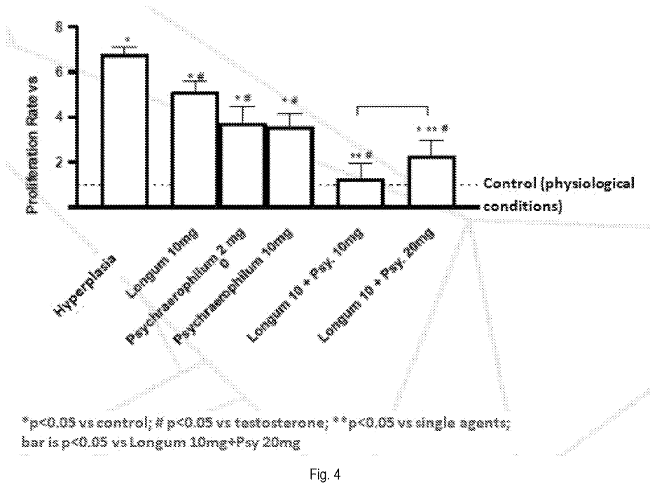

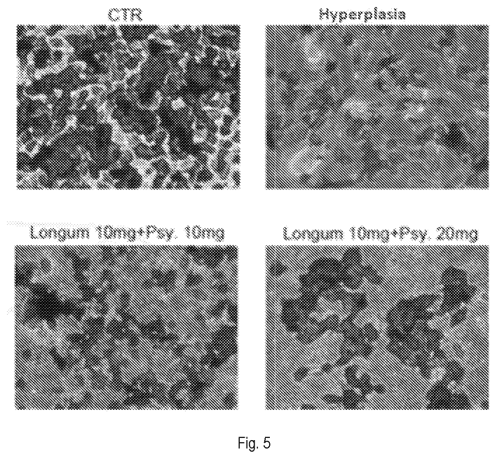

Prostatic analysis after intestinal passage reveals that all probiotics tested are able to counteract the proliferation rate leading to hyperplasia. In particular, The combination of Longum 10 mg+Psy 10 mg and Longum 10 mg+Psy. 20 mg reduced cell volume and, consequently, proliferation activity was reduced compared to the single agents (p<0.05) (FIGS. 4 and 5).

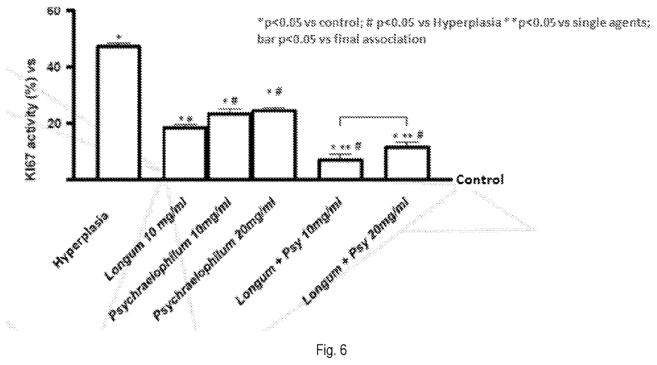

Hyperplasia aggravated prostate function, significantly increased the activity of Ki67, a proliferation marker, leading to cellular distress. Probiotics are able to slow down this process (p<0.05), and the positive effects exerted by Longum and psychaerophilum are amplified due to their cooperative activity on the intestinal epithelium (p<0.05) (FIG. 6).

### Reduction of Oxidative Stress

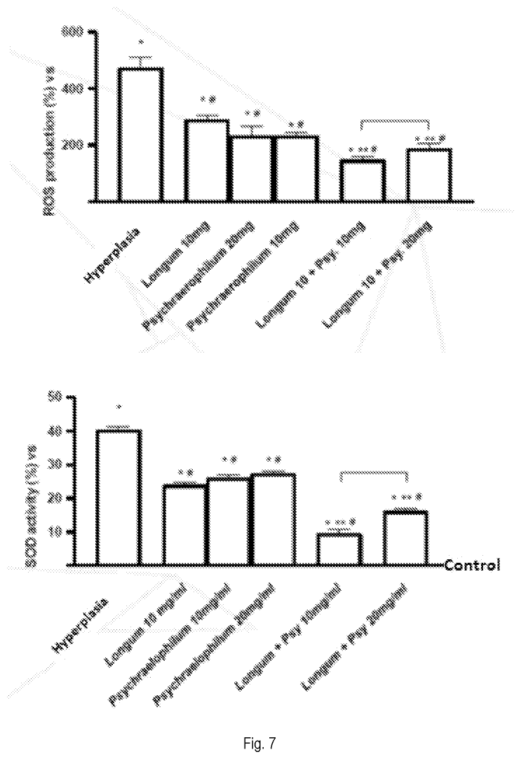

All probiotics alone or combined are able to significantly reduce oxidative stress leading to tissue damage (p<0.05) causing hyperplasia condition confirming the absence of side effects (safety) and the ability to modulate disease progression (FIG. 7).

### Reduction of Inflammation

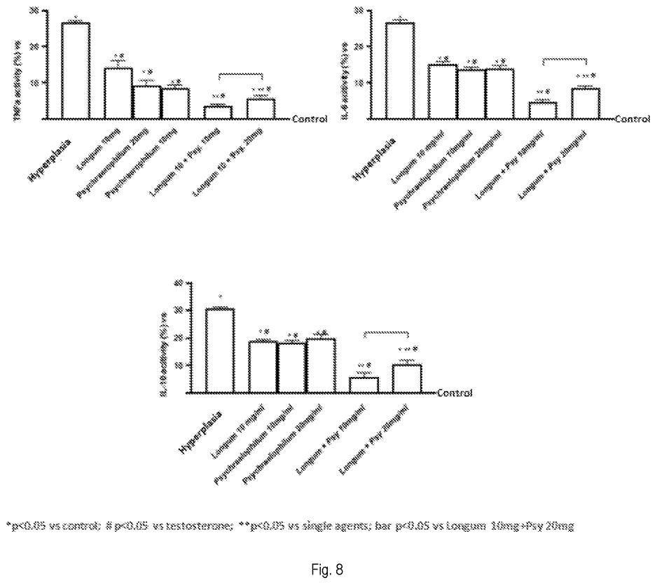

Inflammation analyses show that all probiotics alone or combined are able to significantly reduce the inflammatory condition (p<0.05) of hyperplasia confirming the absence of side effects (safety) and the ability to directly modulate disease progression (FIG. 8).

### Androgen Receptor (AR) Activity

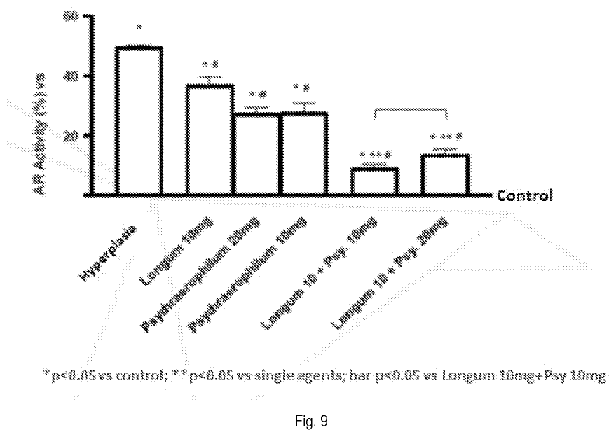

AR hyperactivity is a marker of prostatic hyperplasia; many therapeutic strategies involved inhibition of AR activity to reduce prostate gland size. The association of probiotics maintains normal AR activity by inducing its reduction during hyperplasia (FIG. 9).

### Testosterone Level

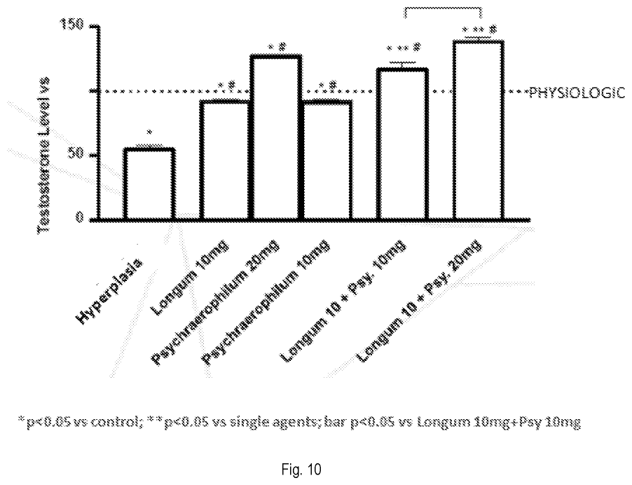

During hyperplasia, the testosterone level is lower due to AR hyperactivity. The association of probiotics improves the testosterone level while maintaining lower AR activity. These data confirm the ability of probiotics to improve prostate condition without side effects (FIG. 10).

### Serotonin Level and Receptor Activity

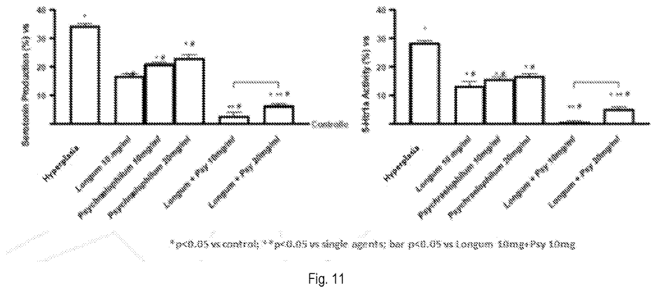

Serotonin is a strong negative regulator of prostate growth. The association of probiotics decreases the level of serotonin by keeping the activity of its receptor (5-Htr1a) low. These data confirm the ability of probiotics to improve prostate condition by inhibiting prostate growth (FIG. 11).

### PSA Level

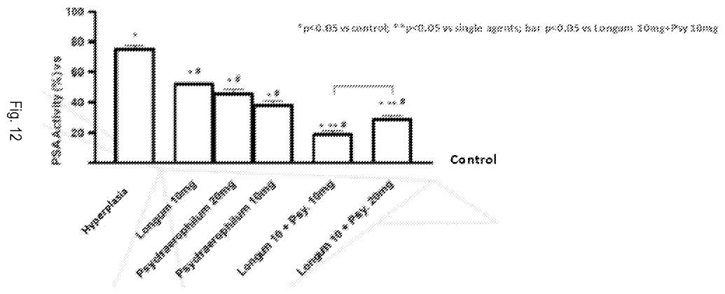

Prostate-specific antigen (PSA) is a sensitive and specific marker for prostate disease in which it is increased. PSA is produced during the hyperplasia condition (p<0.05), but all individual agents are able to reduce this production suggesting their positive role during BPH. In particular, The association between Longum and Psy is able to amplify the positive role of probiotics with the maximum effect exerted by Longum 10 mg+Psy 10 mg (FIG. 12).

The results of the conducted assays showed that the association of the first aspect of the invention is able to reduce prostate cell proliferation, reduces oxidative stress and inflammatory network, reducing the negative consequences of BPH.

In addition, the association improves the function of androgen receptor, testosterone level, and serotonin level, restoring the balance between them to reduce pathology.

All these important effects are believed to be due to SCFAs secreted by probiotics, e.g., butyric acid, which can act as second messengers and activate various mechanisms that are altered during BPH.

It is important to report that the tested strains did not induce any damage on epithelia, confirming their safety properties by enhancing antioxidant mechanisms and anti-inflammatory activity during BPH by also acting on the prostate serotonergic pathway and decreasing proliferation activity.

To confirm the veracity of the data obtained, analyses performed on the molecular pathways involved in the development of BPH were also performed using the standard model of BPH [Perks C M, Zielinska H A, Wang J, et al. Insulin Receptor Isoform Variations in Prostate Cancer Cells. Front Endocrinol (Lausanne) 2016; 7:132].

At the same time, the Standard BPH Model was also used to verify what was observed in the main molecular mechanisms modified by probiotic treatments according to the following method.

### Method: Conventional 3D Model of Prostate Spheroid in an In Vitro Model

A conventional in vitro 3D spheroidal model was created to verify the beneficial effects of the probiotic strain during BPH after oral intake; to date, a validated protocol reported in the literature by Rodriguez-Dorantes et al. has been followed in detail [Allers K, Stahl-Hennig C, Fiedler T. et al. The colonic mucosa-associated microbiome in SIV infection: shift toward Bacteroidetes coincides with mucosal CD4+ T cell depletion and enterocyte damage. Sci Rep. 2020:10, 10887]. The LNCap cell line was used for this purpose because they are androgen-dependent cells; once the cells reached 90% confluence, they were washed with 2 mL of PBS1×, separated with 3 mL of StemPro Accutase (Thermofisher, Waltham, Massachusetts, USA) and centrifuged for 4 min at 300 g. Next, cells were suspended in 1 mL of DMEM-F12 supplemented with 20 ng/ml EGF, 20 ng/ml bFGF, B27 1×, 5 μg/ml insulin, 4 μg/ml heparin (all purchased from Merck Life Science, Rome, Italy). Cells were counted and 2000 cells/mL (3 mL as final volume) were plated in 6-well plates with very low adhesion (Merck Life Science, Rome, Italy) for spheroid formation. The plates were incubated at 37° C. and 5% CO2, and spheroid formation was observed between 2 and 7 days. Every 7 days, 50% of the medium was changed by tilting the plate slightly and holding it in this position for 10 min. Spheroid growth was monitored for 28 days. At the end of time, mature spheroids were treated overnight with 0.5 UM testosterone to mimic BPH in vitro [Huang K, Li W, Chen Y, Zhu J. Effect of PM2.5 on invasion and proliferation of Hela cells and the expression of inflammatory cytokines IL-1 and IL-6. Oncol Lett. 2018; 16 (6): 7068-7073].

### Western Blot of Cell Lysates

Western blot technique was used to analyze the expression of AR and PSA, respectively, according to the following protocol.

### Western Blot Method of Cell Lysates

Spheroids producing both standard and modified BPH models were lysed in ice-cold Complete Tablet Buffer (Roche, Milan, Italy) supplemented with 2 mM sodium orthovanadate, 1 mM phenylmethanesulfonyl fluoride (PMSF; Sigma-Aldrich), a 1:50 mixture of phosphatase inhibitor cocktail (Sigma-Aldrich) and a 1:200 mixture of protease inhibitor cocktail (Calbiochem, San Diego, CA, USA). At the same time, LNCap cells were washed twice with ice-PBS 1× and lysed in ice-cold Ripa Buffer (50 mM Hepes, 150 mM NaCl, 0.1% SDS, 1% TRITON 100×, 1% deoxycholate acid, 10% glycerol, 1.5 mM MgCl2, 1 mM EGTA, 1 mM NaF; all purchased from Merck Life Science, Rome, Italy) supplemented with 2 mM sodium orthovanadate (Na₃VO₄, Merck Life Science, Rome, Italy), 1 mM phenylmethanesulfonyl fluoride (PMSF; Merck Life Science, Rome, Italy) and a 1:100 mixture of protease inhibitor cocktail (Merck Life Science, Rome, Italy). 40 μg of protein from each sample was resolved on 10% SDS-PAGE gels. Polyvinylidene difluoride membranes (PVDF, GE, Healthcare Europe GmbH, Milan, Italy) were incubated overnight at 4° C. with a specific primary antibody: anti-AR (1:250, Santa Cruz Biotechnology) and anti-PSA (1:250, Santa Cruz Biotechnology). Protein expression was normalized and verified by detection of β-actin (1:5000; Merck Life Science, Rome, Italy) and expressed as mean±SD (% vs. control).

### Results

Cell Viability of Caco-2 Cells with Probiotics.

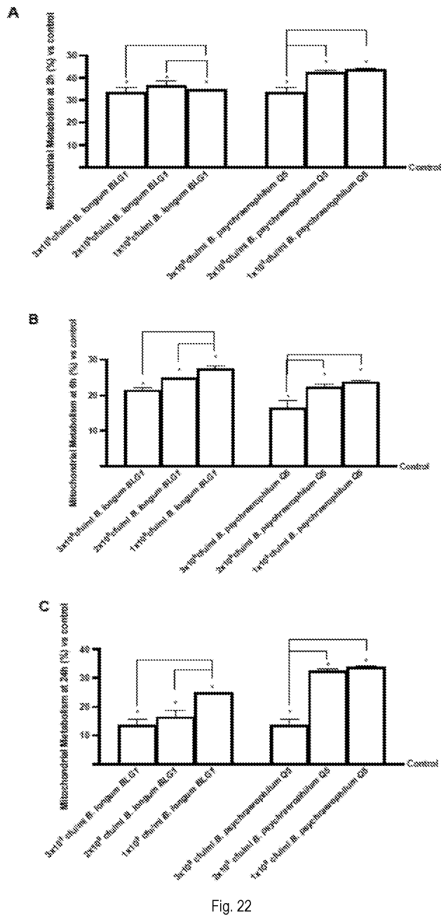

Mitochondrial metabolism of Caco-2 cells was analyzed in a study over time (2 hours, 6 hours and 24 hours) to simulate human uptake time by treating with 1*10⁹, 2*10⁹ and 3*10⁹ CFU/ml of B. longum BLG1 or 1*10⁹ 2*10⁹ and 3*10⁹ CFU/ml of B. psychaerophilum Q5. These test dosages were selected to provide at least 10⁹ live bacterial cells per day following the recommendation provided by the Italian Ministry of Health in the “guidelines on probiotics and prebiotics” revised in March 2018. As shown in FIG. 22, all concentrations tested were able to induce an increase in CaCo-2 cell viability compared to control (p<0.05), excluding any cytotoxic effects. Specifically, with regard to B. longum BLG1, only 1*10⁹ CFU/ml was able to increase mitochondrial well-being compared to control (p<0.05) and, more importantly, compared to both 2*10⁹ CFU/ml and 3*10⁹ CFU/ml, respectively (p<0.05). These results suggest that 1*10⁹ CFU/ml of B. longum BLG1 is the correct dosage. As for B. psychaerophilum Q5, both 2*10⁹ CFU/ml and 1*10⁹ CFU/ml gave satisfactory results as they stimulated mitochondrial metabolism compared with control (p<0.05) and compared with 3*10⁹ CFU/ml (p<0.05). These cellular mechanisms could underlie the probiotic's ability to stimulate intestinal cell well-being. Therefore, 1*10⁹ CFU/ml of B. longum BLG1 and 1*10⁹ CFU/ml and 2*10⁹ CFU/ml of B. psychaerophilum Q5 were also selected to be analyzed in subsequent experiments.

The Effects of B. longum and B. psychaerophilum on the Intestinal Barrier in the In Vitro Model

Considering the results obtained on Caco-2 cells, the regulatory abilities of B. longum BLG1 and B. psychaerophilum Q5 were also studied in a 3D intestinal model in vitro, mimicking the human intestinal barrier complexity. In this context, 1*10⁹ CFU/ml of B. longum BLG1 both 1*10⁹ CFU/ml and 2*10⁹ CFU/ml of B. psychaerophilum Q5 alone and combined were tested from 2 hours to 6 hours, measuring tight junction (TJ) activities at the end of the stimulation time (6 hours).

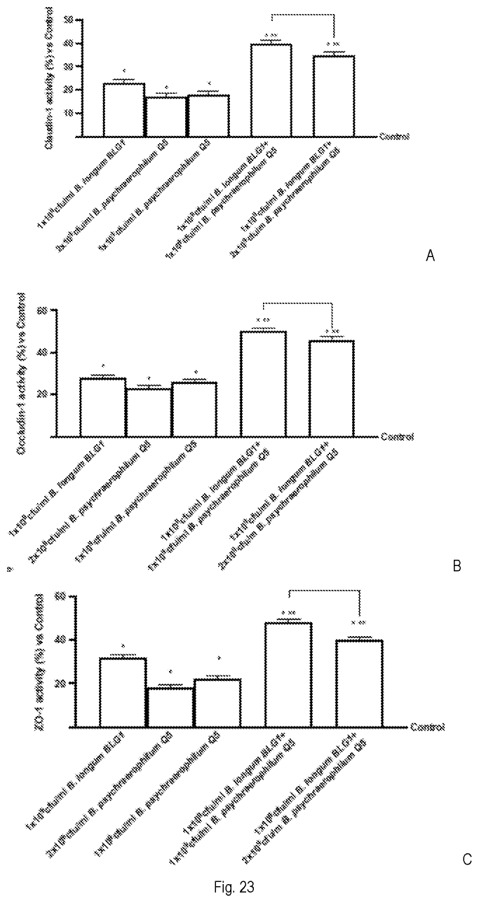

At the end of the stimulation time, zone occludens-1 (ZO-1), claudin-1 and occludin-1 (FIG. 23) confirmed the beneficial effects of all tested probiotic concentrations and the ability to maintain the physiological function of all selected tight junctions (p<0.05). As expected, only 1*10⁹ CFU/ml of B. longum BLG1 plus 1*10⁹ CFU/ml of B. psychraelophilum Q5 exerted a notable effect compared with the individual agents (p<0.05) and, especially, compared with the other combination consisting of 1*10⁹ CFU/ml B. longum BLG1 plus 2*10⁹ CFU/ml B. psychraelophilum Q5 (about 20% ZO-1, about 14% for claudin-1 and about 9% for occludin-1, p<0.05), confirming the regulatory effect of probiotics on the intestinal barrier. From these encouraging results, further experiments were conducted by measuring one of the major SCFA produced over time by B. longum BLG1 and B. psychaerophilum Q5.

In a second aspect, it is an object of the invention at least one bacterial strain selected from the group comprising or alternatively consisting of:
Bifidobacterium bifidum “novaBBF9” deposited at the Deutsche Sammlung von Mikroorganismen und Zellkulturen GmbH (DSMZ) under deposit number DSM 34337, by Probionova SA on Jul. 26, 2022 on Jul. 26, 2022,
Lactobacillus crispatus “novaLCR” deposited at the Deutsche Sammlung von Mikroorganismen und Zellkulturen GmbH (DSMZ) under deposit number DSM 34348, by Probionova SA on Jul. 26, 2022, and
Lactobacillus fermentum “novaLV58” deposited at the Deutsche Sammlung von Mikroorganismen und Zellkulturen GmbH (DSMZ) under deposit number DSM 34340, by Probiotics AG on Jul. 26, 2022;
   and mixtures thereof.

It is also an object of the second aspect of the invention a pharmaceutical or nutraceutical or dietary supplement association or combination comprising or alternatively consisting of two strains of the invention (Association A2):
Bifidobacterium bifidum “novaBBF9” DSM 34337 and Lactobacillus crispatus “novaLCR” DSM 34348; preferably in a weight ratio from 1:10 to 10:1, even more preferably in a weight ratio from 1:5 to 5:1, e.g., from 1:3 to 3:1; or from 1:2 to 2:1; or 1:1, preferably all strains of bacteria are in lyophilized form.
Bifidobacterium bifidum “novaBBF9” DSM 34337 and Lactobacillus fermentum “novaLV58” DSM 34340; preferably in a weight ratio from 1:10 to 10:1, even more preferably in a weight ratio from 1:5 to 5:1, e.g., from 1:3 to 3:1; or from 1:2 to 2:1; or 1:1, preferably all strains of bacteria are in lyophilized form.
Lactobacillus crispatus “novaLCR” DSM 34348 and Lactobacillus fermentum “novaLV58” DSM 34340; preferably in a weight ratio from 1:10 to 10:1, even more preferably in a weight ratio from 1:5 to 5:1, e.g., from 1:3 to 3:1; or from 1:2 to 2:1; or 1:1, preferably all strains of bacteria are in lyophilized form.
   or, a pharmaceutical or nutraceutical or dietary supplement association or combination comprising or, alternatively, consisting of the three strains of the invention: —Bifidobacterium bifidum “novaBBF9” DSM 34337, Lactobacillus crispatus “novaLCR” DSM 34348, and Lactobacillus fermentum “novaLV58” DSM 34340; preferably in a weight ratio of 1:1:2, or 1:2:1, or 2:1:1, or 1:1:1, preferably all strains of bacteria are in lyophilized form.

It is also an object of the second aspect of the invention a pharmaceutical or nutraceutical or dietary supplement composition comprising one of the strains described above or association thereof (A2), optionally together with at least one physiologically and/or pharmaceutically acceptable excipient and/or vehicle.

It is also an object of the second aspect of the invention the use of said bacterial strains and/or mixtures thereof in therapy and, more specifically, in the curative or prophylactic treatment of gynecological diseases, preferably diseases associated with alterations in myometrial trophism and endocrine and metabolic disorders, such as polycystic ovary syndrome (PCOS), endometrial hyperplasia, and endometriosis.

It is also an object of the second aspect of the invention the use of the compositions of the invention in therapy and, more specifically, in the curative or prophylactic treatment of gynecological diseases, preferably diseases associated with alterations in myometrial trophism and endocrine and metabolic disorders, such as polycystic ovary syndrome (PCOS), endometrial hyperplasia, and endometriosis.

Preferably, the second aspect refers to a composition comprising from 1 to 100 mg of Bifidobacterium bifidum novaBBF9, from 1 to 100 mg of Lactobacillus crispatus novaLCR, and from 0.1 to 100 mg of Lactobacillus fermentum novaLV58, preferably from 5 mg to 50 mg of Bifidobacterium bifidum novaBBF9, from 5 mg to 50 mg of Lactobacillus crispatus novaLCR, and from 1 mg to 50 mg of Lactobacillus fermentum novaLV58, even more preferably from 10 mg to 40 mg of Bifidobacterium bifidum novaBBF9, e.g. 20 mg or 30 mg of Bifidobacterium bifidum novaBBF9, from 10 mg to 40 mg of Lactobacillus crispatus novaLCR, e.g. 20 mg or 30 mg of Lactobacillus crispatus novaLCR, and from 5 mg to 40 mg of Lactobacillus fermentum novaLV58, e.g. 20 mg or 30 mg of Lactobacillus fermentum novaLV58, preferably all strains of bacteria are in lyophilized form.

Preferably, a composition may comprise from 5 mg to 20 mg of Bifidobacterium bifidum novaBBF9, from 5 mg to 20 mg of Lactobacillus crispatus novaLCR, and from 3 mg to 10 mg of Lactobacillus fermentum novaLV58; preferably all strains of bacteria are in lyophilized form.

Preferably, the second aspect of the invention relates to a composition comprising 10 mg of Bifidobacterium bifidum novaBBF9, 10 mg of Lactobacillus crispatus novaLCR, and 5 mg of Lactobacillus fermentum novaLV58, preferably all strains of bacteria are in lyophilized form.

Compositions of the second aspect of the invention may comprise one or more pharmaceutically acceptable excipients and/or vehicles.

Preferably, the strains Bifidobacterium bifidum novaBBF9 and Lactobacillus crispatus novaLCR are administered in daily doses ranging from 1.0×10⁶ CFU/day to 1.0×10¹² CFU/day, preferably from 1.0×10⁸ CFU/day to 1.0×10¹⁰ CFU/day, e.g., about 1.0×10⁹ CFU/day, while the Lactobacillus fermentum novaLV58 strain is administered in daily doses ranging from 0.5×10⁶ CFU/day to 0.5×10¹² CFU/day, preferably from 0.5×10⁸ CFU/day to 0.5×10¹⁰ CFU/day, e.g., about 0.5×10⁹ CFU/day (CFU=Colony Forming Units).

Compositions can be administered once or several times a day, preferably once a day.

Strains of the second aspect of the invention may be present in the composition of the second aspect of the invention as live and viable bacteria, dead bacteria or cellular components, cell extracts, lysates or tyndallized thereof.

According to an embodiment, the at least one physiologically and/or pharmacologically acceptable excipient may include prebiotics. According to an embodiment, the at least one physiologically and/or pharmacologically acceptable excipient may include a monosaccharide, a disaccharide, a polysaccharide, soluble fiber and/or insoluble fiber, GOS, FOS, inulin, maltodextrin, and mixtures thereof.

According to an embodiment, the at least one physiologically and/or pharmacologically acceptable excipient may include partially hydrolyzed guar gum (PHGG).

According to different embodiments, the physiologically and/or pharmacologically acceptable excipient could be selected from the group consisting of: inulin, a fructooligosaccharide (FOS), maltodextrin and mixtures thereof.

Compositions according to the first aspect of the invention are preferably compositions for oral use, advantageously in capsule form. However, other pharmaceutical forms may be used.

The strains of the second aspect of the invention have been the subject of numerous experimental assays, reported below.

During these assays, the Applicant observed that particularly when associated, the association A2 is able to stimulate the gut/myometrial axis while maintaining myometrial function. In addition, the association is able to improve myometrial activity by modulating cell cycle progression.

The assays conducted and the results obtained with the association A2 of the second aspect of the invention will be shown below, with reference to the attached Figures.

### Experimental Section—Myometrial Activity

### Legend

L. Crispatus=Lactobacillus crispatus novaLCR
L. Fermentum=Lactobacillus fermentum novaLV58
B Bifidum=Bifidobacterium bifidum novaBBF9
CFU=colony-forming units

A dose-response study was conducted on an in vitro model using PHM1-41 cells to evaluate the safety of different probiotics and substances, as follows:
Lactobacillus crispatus novaLCR in the range from 10 mg/ml to 50 mg/ml (corresponding to 1×10⁹-5×10⁹ CFU)
Lactobacillus fermentum novaLV58 in the range from 10 mg/ml to 50 mg/ml (corresponding to 1×10⁹-5×10⁹ CFU)
Bifidobacterium bifidum novaBBF9 in the range from 10 mg/ml to 50 mg/ml (corresponding to 1×10⁹-5×10⁹ CFU)
a Lactobacillus Gasseri in a range from 5 mg/ml to 50 mg/ml (corresponding to 1×10⁹1×10⁸ CFU)
d-chiro-inositol (DCI) in the range from 150 mg/ml to 300 mg/ml
inositol in a range from 18 μg/ml to 1000 mg/ml.

All substances were prepared directly in the stimulation medium.

The best concentration of each probiotic (based on conversion from CFU to mg/ml per day) was maintained for all subsequent experiments.

Based on the data obtained, the best concentration of each substance was tested for intestinal barrier or modulation of mediators released by probiotics. In this context, a gut/myometrium co-culture model was realized by seeding intestinal CaCo-2 cells in the apical part of the Transwell® system and stimulating the cells with L. crispatus 10 mg/ml, L. fermentum 5 mg/ml, B. bifidum 10 mg/ml, alone and in combination. The metabolized samples of intestinal cells were used to stimulate myometrial epithelium (PHM1-41 cells) placed in the basolateral compartment for 48 hours to analyze mitochondrial metabolism. Further experiments were then performed to evaluate myometrial cell proliferation (Crystal Violet), ROS and oxytocin production, TNF-α activity, and to assess the influence of ovarian activity by analyzing the level of FSH and LH, which are mainly involved in the fertility program.

### Materials and Methods

### Preparation of Solutions

Lyophilized L. Crispatus, L. fermentum and B. Bifidum were dissolved directly in PBS1× to prepare different concentrations for testing. L. Crispatus and L. Fermentum were tested in the range from 50 mg/ml to 10 mg/mL, and B. Bifidum from 200 g/mL to 5 mg/mL. The concentrations of each probiotic correspond to human oral use as shown in Table 1.

TABLE 1
Sample concentration range

| | | | Low dose | Low dose | High Dose | High Dose |
|---|---|---|---|---|---|---|
| Sample | Genus | Species | (CFU) | (mg) | (CFU) | (mg) |
| Probiotic | Lactobacillus | Crispatus | 1 × 10⁹ | 10 | 5 × 10⁹ | 50 |
| Probiotic | Lactobacillus | Fermentum | 0.5 × 10⁹ | 5 | 5 × 10⁹ | 50 |
| Probiotic | Bifidobacterium | Bifidum | 1 × 10⁹ | 10 | 5 × 10⁹ | 50 |
| Probiotic | Lactobacillus | Gasseri | 1 × 10⁹ | 5 | 1 × 10¹⁰ | 50 |
| Inositol | | | | 125 | | 1000 |
| DCI | | | | 150 | | 300 |

Inositol and d-chiroinositol (DCI) were dissolved in PBS1× in a range normally used in commercial formulations (from 150 mg/ml to 1000 mg/mL).

### Cell Culture

Human intestinal epithelial cells, CaCo-2, purchased from the American Type Culture Collection (ATCC), were cultured in Dulbecco's Modified Eagle's Medium/Nutrient F-12 Ham's (DMEM-F12, Merck Life Science, Rome, Italy) containing 10% FBS, 2 mM L-glutamine and 1% penicillin-streptomycin and maintained in an incubator at 37° C. and 5% CO2. Cells for the experiments were used at passage numbers between 26 and 32 to preserve the integrity of paracellular permeability and transport properties while maintaining similarity to the intestinal absorption mechanism following oral intake in humans.

The PHM1-41 cell line was derived from pregnant human myometrium and maintained in Dulbecco's Modified Eagle's Medium (DMEM, Merck Life Science, Rome, Italy) supplemented with 10% FBS, 2 mM L-glutamine and 1% penicillin/streptomycin and incubated at 37° C. at 5% CO2 and 95% humidity.

CHO-K1 (Chinese Hamster Ovary) cells, a widely used model to evaluate the role of probiotics in the fertility program, were cultured in Dulbecco's modified Eagle's medium: Nutrient Mixture F-12 (DMEM-F12; Sigma, Milan, Italy) supplemented with 10% fetal bovine serum (FBS, Sigma, Milan, Italy), 2 mM glutamine and 1% penicillin/streptomycin (Sigma, Milan, Italy) and incubated at 37° C., 5% CO2 and 95% humidity.

### MTT Testing

After each stimulation, cells were washed with PBS1× and incubated with DMEM without phenol red and FBS containing 1% MTT dye (MTT-Based In Vitro Toxicology Assay Kit; Sigma-Aldrich) for 2 hours at 37° C. and 5% CO2. Cell viability was determined by measuring absorbance with a spectrophotometer (Infinite 200 Pro MPlex, Tecan) at 570 nm with correction at 690 nm and calculated by comparing the results with control cells (100% viable).

### Cristal Violet

Cell proliferation was studied by Cristal Violet staining as reported in the literature. After treatment, cells were washed with PBS1× and fixed with 1% glutaraldehyde in PBS1× (% v/v) for 15 min at room temperature. 100 μl of 0.1% Cristal violet in aqueous solution (% v/v) was added to each well for 20 min at room temperature. The cells were washed again and 100 μl of 10% acetic acid in PBS1× (% v/v) was added to solubilize them; then, absorbance was measured with a spectrophotometer (Infinite 200 Pro MPlex, Tecan) at 595 nm. Cell number was calculated by comparing the data with control cells and normalized against untreated cells examined at time zero.

### Gut/Myometrium Co-Culture Model

CaCo-2 cells were seeded, as previously described, in Transwell® inserts at a density of 20000 cells/cm2 and cultured for 21 days in complete medium, as previously reported. Then, 3-5 days before maturity, PHM1-41 cells were added to the basolateral side of the insert system at a concentration of 50000 cells/600 μL, and the co-culture was maintained in complete medium for three days. The medium was changed starting from the apical side of the wells and incubated for up to 2 hours, then treatments were performed. L. crispatus 10 mg/ml, L. Fermentum 5 mg/ml and B. Bifidum 10 mg/ml, alone and combined, were added in the apical part from 2 h to 6 h, mimicking intestinal absorption. At the end of the time, basolateral medium was collected to measure probiotic metabolization using the butyric acid assay kit. At 6 h after stimulation, the transwell insert was removed to perform subsequent tests on PHM1-4, such as analysis of proliferation, oxidative stress and inflammatory processes, hormonal and molecular pathways involved.

### Gut/Ovarian Co-Culture Model

CaCo-2 cells were seeded, as previously described, in Transwell® inserts at a density of 20000 cells/cm2 and cultured for 21 days in complete medium, as previously reported. Then, 3-5 days before maturity, CHO-K1 cells were added to the basolateral side of the insert system at a concentration of 60000 cells/600 μL, and the co-culture was maintained in complete medium for three days. The medium was changed starting from the apical side of the wells and incubated for up to 2 hours, then treatments were performed. L. crispatus 10 mg/ml,
L. Fermentum 5 mg/ml and B. Bifidum 10 mg/ml, alone and combined, were added to the apical environment from 2 h to 6 h, simulating intestinal absorption. At 6 h after stimulation, the Transwell® insert was removed to perform the subsequent FSH and LH production test.

### Absorption Analysis

Cells were stimulated with the selected agents added to the apical part of the Transwell® system in a time-dependent study (from 2 h to 6 h), to test at each time point the intestinal uptake or bioavailability using the fluorescein 0.04%, marker dye of transepithelial transport. Fluorescence was detected with a fluorescence spectrophotometer (Infinite 200 Pro MPlex, Tecan, Cernusco sul Naviglio, MI, Italy) at excitation/emission wavelengths of 490/514 nm.

### Quantification of Butyric Acid

To quantify probiotic metabolites, butyric acid production was analyzed using the ELISA kit (Cloud-Clone, Wuhan) according to the manufacturer's instructions. The absorbance of each sample was measured after the addition of the stop solution at 450 nm using a plate reader (Infinite 200 Pro MPlex, Tecan), and OD was interpolarized with a standard curve (from 10,000 pg/mL to pg/ml), expressing the data as the mean (pg/ml) against the control.

### ROS Production

The rate of superoxide anion release was measured using a standard protocol based on cytochrome C reduction. In both treated and untreated cells, 100 μL of cytochrome C and, in another sample, 100 μL of superoxide dismutase were added for 30 min in an incubator (all substances were provided by Sigma-Aldrich). Absorbance in the culture supernatants was measured at 550 nm with a spectrometer (Infinite 200 Pro MPlex, Tecan), and 02 was expressed as the mean±SD (%) of nano moles of reduced cytochrome C per microgram of protein compared with the control.

### TNF-α ELISA Kit

TNF-α concentration in basolateral medium was determined using the TNF-α ELISA kit (Merck Life Science, Rome, Italy) according to the manufacturer's instructions. The absorbance of each well was measured after addition of the stop solution at 450 nm using a plate reader (Infinite 200 Pro MPlex, Tecan).

### LH ELISA Kit

Luteinizing hormone (LH) concentration was determined with the Human LH (Luteinizing Hormone) ELISA kit (FineTest) according to the instructions. Briefly, 100 uL of each sample was added to each well and the plate was incubated at 37° C. for 90 minutes. At the end of incubation, the material in each well was removed and the wells were washed twice with wash buffer. 100 uL of biotin-labeled antibody working solution was added to the wells, and the plate was incubated at 37° C. for 60 minutes. At the end of incubation, the solution in each well was removed and the wells were washed three times with wash buffer. Then, 100 μL of SABC working solution was added to each well and the plate was incubated at 37° C. for 30 minutes. At the end, the wells were washed five times and 90 uL of TMB substrate was inserted into each well. After 10-20 minutes, 50 uL of stop solution was introduced into each well and the plate was immediately read at 450 nm using a plate reader (Infinite 200 Pro MPlex, Tecan). A standard curve was plotted relating the color intensity to the concentration of the standard (range from 0.938 to 60 mlU/mL).

### FSH ELISA Kit

Follicle-stimulating hormone (FSH) concentration was determined with the Human FSH (Follicle-Stimulating Hormone) ELISA kit (FineTest) according to the instructions. Briefly, 100 uL of each sample was added to each well and the plate was incubated at 37° C. for 90 minutes. At the end of the incubation time, the material in each well was removed and the wells were washed twice with wash buffer. 100 μL of biotin-labeled antibody working solution was added to the wells, and the plate was incubated at 37° C. for 60 minutes. At the end of incubation, the solution in each well was removed and the wells were washed three times with wash buffer. Then, 100 μL of SABC working solution was added to each well and the plate was incubated at 37° C. for 30 minutes. At the end, the wells were washed five times and 90 μL of TMB substrate was inserted into each well. After 10-20 minutes, 50 μL of stop solution was introduced into each well and the plate was immediately read at 450 nm using a plate reader (Infinite 200 Pro MPlex, Tecan). A standard curve was plotted relating the color intensity to the concentration of the standard (range from 1.563 to 100 mlU/mL).

### Oxytocin ELISA Kit

Oxytocin (OT) concentration was determined by Oxytocin ELISA Kit (Cayman Chemical Company; Ann Arbor, MI, USA) according to the instructions. This assay is based on competition between oxytocin and oxytocin conjugated to acetylcholinesterase (AChE) (oxytocin tracer) for a limited amount of polyclonal oxytocin antiserum. Since the concentration of oxytocin tracer is kept constant while the concentration of oxytocin varies, the amount of oxytocin tracer able to bind to the polyclonal oxytocin antiserum will be inversely proportional to the concentration of oxytocin in the well. The plate was washed to remove unbound reagents and then Ellman's Reagent was added to the wells. The enzyme reaction product was read at a wavelength between 405 and 420 nm. A standard curve is plotted relating the intensity of the color to the concentration of the standard (range from 15.625 to 1000 pg/mL).

### AKT/ERK ELISA Kit

AKT/ERK activation was measured by the InstantOne™ ELISA method (Thermo Fisher, Milan, Italy) on chondrocyte lysates as reported in the literature. The strips were measured by a spectrometer at 450 nm (Infinite 200 Pro MPlex, Tecan). Results were expressed as mean absorbance (%) compared with control.

### Cyclin D1 ELISA Kit

Cyclin D1 production was determined using the ELISA kit (MyBiosource, San Diego, CA, USA), which analyzes cyclin D1 in cell lysates, according to the manufacturer's instructions. Briefly, 100 μl of each sample was added to the wells and the plate was incubated for 80 min at 37° C.; then the wells were washed 3 times with 200 μl of wash solution and 100 μl of biotinylated antibody was added before incubating the plate for 50 min at 37° C. Then the wells were washed again 3 times with 200 μl of wash solution before adding 100 μl of Streptavidin-HRP working solution for 50 min at 37° C. Before adding 90 μl of TMB substrate solution to each well, the wells were washed again for 5 times. Finally, after 50 minutes of incubation at 37° C., 50 μl of Stop reagent was added to each well. The samples were analyzed with a spectrometer (Infinite 200 Pro MPlex, Tecan) at 450 nm. Concentration is expressed as ng/ml relative to a standard curve (range 0.32 to 20 ng/mL), and results are expressed as percent (%) relative to control (line 0).

### Annessin V ELISA Kit

Annexin V production was determined using the ELISA kit (Abcam, Cambridge, UK), which analyzes Annexin V in cell supernatants, according to the manufacturer's instructions. Briefly, 50 μl of standards or samples were added to each well with 50 μl of antibody cocktail before incubation of the plate for 1 hr in shaking. Next, each well was washed 3 times with 350 μl of 1× wash buffer before incubation of the plate for 10 min in the dark with 100 μl of TMB development solution in each well. Finally, 100 μl of stop solution was added to each well and the samples were analyzed with a spectrometer (Infinite 200 Pro MPlex, Tecan) at 450 nm. Concentration is expressed in ng/ml relative to a standard curve (range 46.9 pg/mL-3000 pg/mL) and results are expressed as percent (%) relative to control (line 0).

### MAGT1 ELISA Kit

MAGT1 production was determined using the ELISA kit (MyBioSource, Vancouver, Canada) that analyzes MAGT1 in cell lysates, according to the manufacturer's instructions. Samples were analyzed with a spectrometer (Infinite 200 Pro MPlex, Tecan) at 450 nm. Concentration is expressed as ng/ml versus a standard curve (range from 0.25 ng/ml-8 ng/ml), and results are expressed as percent (%) versus control (line 0).

### PAX8 ELISA Kit

PAX8 production was determined using the ELISA kit (MyBiosource, San Diego, CA, USA) that analyzes PAX8 in cell lysates, according to the manufacturer's instructions. Samples were analyzed with a spectrometer (Infinite 200 Pro MPlex, Tecan) at 450 nm. Briefly, 100 μl of each sample or standard was added to each well and the plate was incubated for 1 h at 37° C. Then the wells were washed 3 times with wash buffer and 100 μl of HRP-Streptavidin conjugate was added before incubating the plate for 30 min at 37° C. After 5 washes with wash buffer, 90 μl of TMB substrate was added to each well and the plate was incubated for 15-30 min at 37° C. Finally, 50 μl of stop solution was added to each well and the absorbance of the samples was read at 450 nm with a spectrometer (Infinite 200 Pro MPlex, Tecan). Concentration is expressed in ng/ml relative to a standard curve (range from 15.6 to 1000 pg/mL) and results are expressed as percent (%) relative to control (line 0).

### Statistical Analysis

Results are expressed as mean±SD of at least 5 biological replicates for each experimental protocol, and each replicate was repeated 3 times for each experimental protocol. Statistical comparisons between groups were performed using one-way ANOVA with Bonferroni's post hoc test or Mann-Whitney's U test, as appropriate, using GraphPad Prism 5 (GraphPad Software, La Jolla, CA, USA). p<0.05 was considered statistically significant. All densitometric analysis data were normalized to control values (defined as 1). All other data from each experimental protocol were normalized to the percent control values (defined as 0%).

### Results

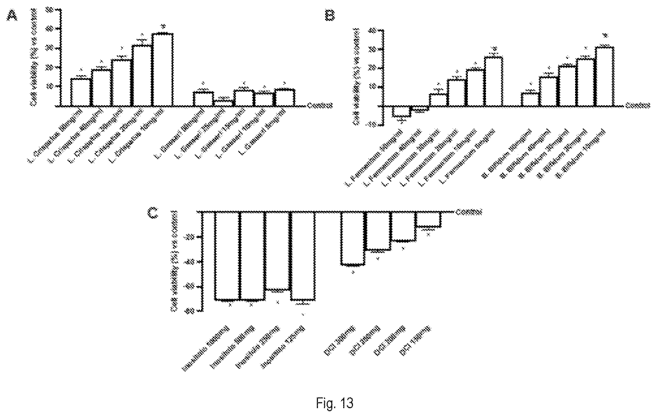

PHM1 cells were used to analyze the interaction between the microbiome and myometrium using different probiotics, combined in a new formulation, that could improve female infertility. A 24-hour dose-response study was then performed to evaluate the best concentration to be used in the final formulation. The range of concentrations was tested based on the literature. Since several treatments for PCOS have been formulated using inositol and d-chiroinositol (DCI), these compounds were also tested in a range normally used in commercial formulations. As shown in FIG. 13, all probiotics were able to improve cell viability compared with the control (p<0.05); in particular, L. crispatus, L. fermentum and B. Bifidum were able to maintain cellular well-being compared with all agents tested (p<0.05). As for Inositol and DCI, none of the tested substances induced the desired results, only DCI 150 mg, which reported less harmful results and was therefore included in the combination. Based on the results obtained, the hypothesized concentrations are L. Crispatus 10 mg/ml+L. Fermentum 5 mg/ml+B. Bifidum 10 mg/ml (association A2) and L. Crispatus 10 mg/ml+L. Fermentum 5 mg/ml+B. Bifidum 10 mg/ml+DCI 150 mg (association A2+DCI 150 mg) (FIG. 13).

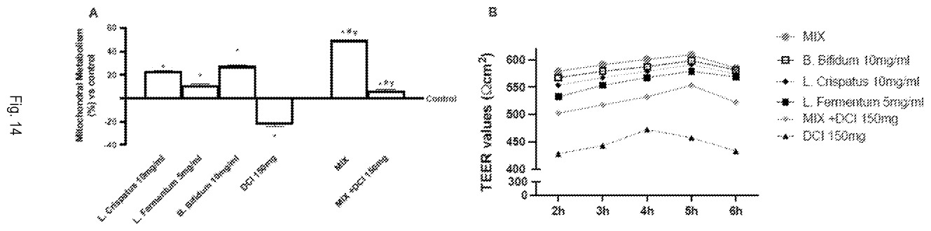

Before conducting further analysis, investigations were conducted to obtain more information on physiological absorption in an EMA- and FDA-validated intestinal barrier model. For this purpose, both mitochondrial metabolism and absorption were analyzed by testing B. Bifidum 10 mg/ml, L. crispatus 10 mg/ml, L. fermentum 5 mg/ml, and DCI 150 mg/ml, alone and in combination, over 6 h (maximum intestinal absorption time) to demonstrate that all substances are able to exert a beneficial effect without altering intestinal cells, excluding any irritability to confirm the safety of the association A2. As shown in FIG. 2, cell viability shows an increase in mitochondrial metabolism for all agents tested compared with the control, although the greatest effect (p<0.05) was observed with the combination of all probiotics. In contrast, the association A2+DCI 150 mg dramatically reduced intestinal cell viability (approximately fourfold, p<0.05), suggesting that the addition of DCI might alter the activity of probiotics. Accordingly, TEER analysis was performed to confirm the integrity of the intestinal monolayer. The reported TEER values are 500±52.9!*cm2 for intestinal CaCo-2 cells according to the literature suggesting that the cells form an intact monolayer induced by the treatments (FIG. 14).

Further analyses were conducted using the same model, analyzing two main aspects of the bioavailability of probiotics: permeability and metabolites produced. As demonstrated in FIG. 3, the intestinal passage analysis showed that all the selected agents are able to cross the intestinal epithelium, confirming the safety data already obtained. In particular, the selected combination increases intestinal passage, suggesting that the probiotic formulation is able to directly reach the target organ, more efficiently than the individual agents and, especially, than the association A2+DCI 150 mg (p<0.05). The same results were also obtained by analyzing the metabolite of probiotics (butyric acid, showing that the ability of probiotics to release short-chain fatty acids (SCFAs) and influence sex hormone secretion is controlled by the A2 association, which amplified the effects exerted by the individual agents (p<0.05). Again, the association A2+DCI 150 mg does not contribute to proper intestinal passage, suggesting that DCI should not be relevant to myometrial homeostasis (p<0.05).

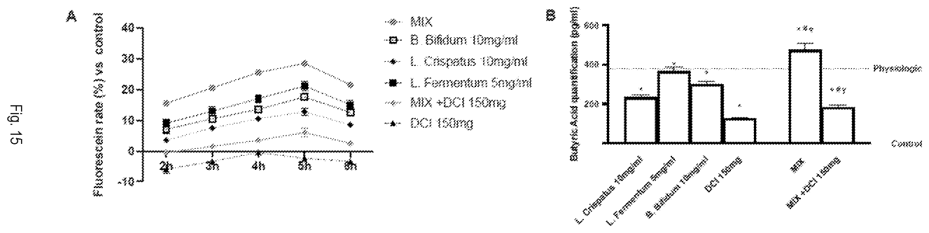

All these data on intestinal absorption are important for understanding the behavior of the formulation within the intestinal compartment: in fact, the association A2 stimulates proper passage of the intestinal barrier, improving intestinal barrier functions and reaching the plasma environment, partly due to the synergistic activity exerted by the selected probiotics by enhancing their bioavailability (FIG. 15).

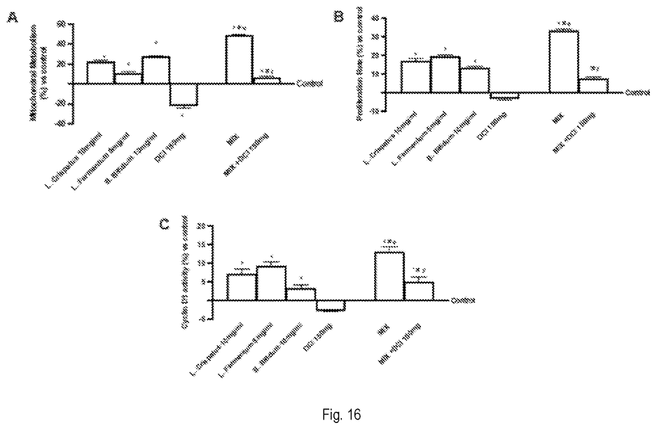

Given this background, it becomes important to consider the direct gut-myometrial effect in a 24-hour co-culture model of myometrial alteration. Therefore, as shown in FIG. 16, analysis of the myometrium after intestinal passage reveals that all probiotics tested are able to enhance the biological activity of the myometrium during myometrial alteration. Our results showed that the combination L. crispatus 10 mg/ml+L. fermentum 5 mg/ml+B. Bifidum 10 mg/ml (referred to as association A2) improved mitochondrial metabolism and, consequently, proliferation activity by amplifying the effects exerted by the individual agents (p<0.05). Specifically, analysis of the mitochondrial metabolism of myometrial cells, in contact with intestinal cells, confirms the hypothesis that the selected combination is able to increase cell viability compared with the single agents (approximately 1.25 times more than L. crispatus; 3 times more than L. fermentum; and 1 time more than B. Bifidum), confirming that it is more effective in increasing cell metabolism, suggesting that it may also play an important role in cell proliferation. In fact, as shown in panel B, proliferation activity is enhanced after treatment with the single agents (p<0.05), but the synergistic effect, obtained when all probiotics are combined, produces an increase in proliferative activity (about 1.66 times more than L. Crispatus; 1 time more than L. Fermentum and 2 times more than B. Bifidum) also confirmed by analysis of cyclin D1, a cell cycle regulatory protein that is required for proliferative activity. In contrast, the association A2+DCI 150 mg produced no significant effect on proliferation analysis (p<0.05), confirming the minor role of DCI in the formulation (FIG. 16).

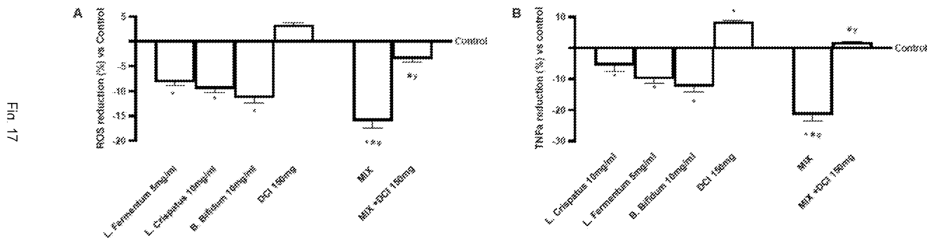

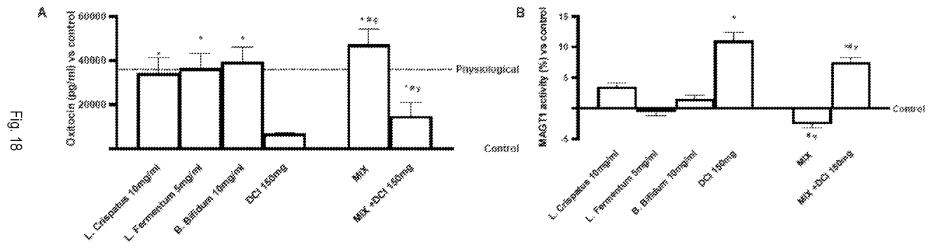

To demonstrate that the increase in proliferative capacity is not due to an alteration of myometrial homeostasis, additional experiments were conducted during myometrial alteration by analyzing ROS production and TNFa activity. As shown in FIG. 5, analysis of oxidative stress (panel A) and inflammation (panel B) shows that all concentrations tested did not induce ROS production and did not result in any inflammatory phenomena. All probiotics combined exerted the maximum effect, confirming the absence of side effects. In contrast, the association A2+DCI 150 mg did not alter ROS production (p<0.05) but induced a slight increase in inflammatory levels, which remained below normal physiological ranges, confirming the controversial role of DCI (FIG. 17). Since all these findings on myometrial epithelia revealed very encouraging data, further experiments were conducted to evaluate the hormonal activity that regulates myometrial function. In particular, several results suggest that oxytocin may be involved in a dual role: in the induction of myometrial contractility and in the control of secretion of some anterior pituitary hormones. In more detail, regarding the contractile role of oxytocin, it plays an essential role in the mechanisms of myometrial contractility through the interaction between myosin and actin, independent of changes in intracellular calcium and magnesium concentrations in smooth muscle tissue. Therefore, the hypercontracting activity of myometrium can cause various physiological changes such as hypertrophy, hyperplasia, contraction, and apoptosis, which are critical for fertility activity. Therefore, to rule out hypercontraction of the myometrium, the level of oxytocin and MAGT1, a magnesium transporter implicated in maintaining intracellular magnesium levels, were observed. In addition, as shown in FIG. 18, results showed that the combination of L. Crispatus 10 mg/ml+L. Fermentum 5 mg/ml+B. Bifidum 10 mg/ml maintained oxytocin and MAGT1 expression at basal level better than the DCI formulation (p<0.05), confirming the active role of the association A2 in myometrial relaxation. These results are important because association A2 is able to maintain the proper balance between calcium and magnesium movement, inhibiting spontaneous myometrial contractility (FIG. 18).

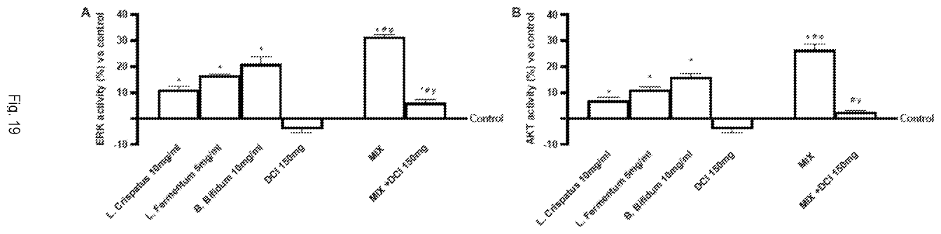

To elucidate the mechanisms underlying the observed results, the selective biomarkers involved in maintaining myometrial homeostasis were analyzed by ELISA kits (FIG. 7A). The ERK/MAPK activity induced by all probiotics combined was higher than that of the single agents (p<0.05), supporting better results regarding myometrial viability and proliferation capacity. In addition, AKT expressions (FIG. 7B) confirmed the role of the new formulation in myometrial activity compared with the control (p<0.05) and compared with the single agents (about 4-fold higher than L. crispatus; 60% compared with L. fermentum and 40% compared with B. Bifidum, p<0.05), confirming its greater influence. Again, the role of DCI was investigated and, as expected, the association A2+150 mg ws unable to maintain the balance of both kinases supporting its controversial effects (p<0.05) (FIG. 19).

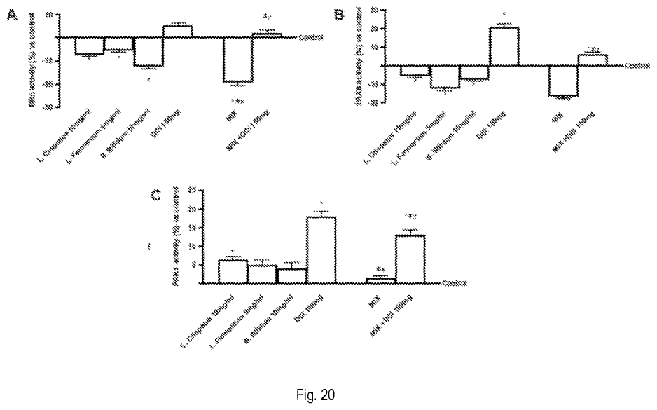

In addition, the major intracellular pathways involved in myometrial dysfunction were analyzed with an ELISA kit to confirm the beneficial effects of the new formulation. Based on this, Erβ, which could be clinically useful in predicting hormonal imbalances and treating inflammatory conditions associated with menopause, infertility, or myometrial dysfunction, PAX8, which is frequently upregulated and functionally essential in an important subset of ovarian cancers leading to infertility, and PAK-1, which is a key member of the CDC42/PAK1 signaling pathway involved in the regulation of cell cycle proliferation and apoptosis during PCOS, were analyzed. As shown in FIG. 20, all tested agents were able to modulate the selected markers (p<0.05), in particular, the association A2 amplified the effects exerted by individual agents confirming the data already obtained on the improvement, safety and maintenance of myometrial function (p<0.05). However, the addition of DCI in the formulation slightly attenuated the alteration observed during myometrial dysfunction without inducing any significant improvement, again confirming that this molecule does not induce beneficial effects on the myometrium and does not interact directly with probiotics (FIG. 20).

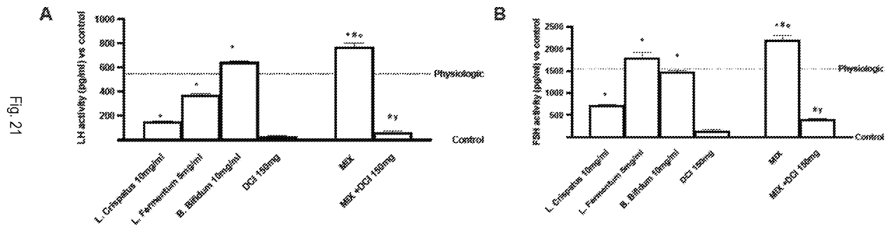

Finally, because LH and FSH play complementary roles in follicle development and ovulation through a complex interaction in the hypothalamus, anterior pituitary, reproductive organs, and oocytes, further experiments were conducted to analyze the effects of association A2 and association A2+DCI 150 mg on ovarian cells. Alteration of gonadotropin production or action causes a relative or absolute deficiency of LH and FSH that impairs gametogenesis and gonadal steroid production, thereby reducing fertility. In women, LH and FSH deficiency is a spectrum of conditions with different functional or organic causes, characterized by low or normal gonadotropin levels and low estradiol levels; the determinants of reduced FSH and LH action are associated with reduced response to ovarian stimulation. Therefore, the analysis of these two hormones on ovarian cells supported the importance of the gut/brain axis on the regulation of fertility homeostasis; as shown in FIG. 9, the association of probiotics can improve the related fertility hormones (FSH and LH) (p<0.05) compared with control. The evidence showed that the combination of the selected probiotics improved hormone secretion better than the single agent (p<0.05) and than the association A2+DCI 150 mg (about 6-fold more with regard to LH and about 3-fold more with regard to FSH), again showing that the presence of DCI does not improve the activity of probiotics (p<0.05). Furthermore, these data support the findings reported in the literature on the importance of supplementation of recombinant LH and FSH to improve hormone levels during fertility programs, as these cells naturally produce these hormones after stimulation with butyric acid (FIG. 21).

1. A pharmaceutical or nutraceutical or dietary supplement composition comprising or, alternatively, consisting of:
Bifidobacterium psychraerophilum “Q5,” deposit number DSM 33131; and
Bifidobacterium longum subsp. longum “novaBLG1,” deposit number DSM 34338;
and, optionally, at least one physiologically and/or pharmaceutically acceptable or food-grade excipient and/or vehicle.

2. The composition according to claim 1, wherein said composition comprises or, alternatively, consists of Bifidobacterium psychraerophilum “Q5” DSM 33131
and Bifidobacterium longum subsp. longum “novaBLG1” DSM 34338, in a weight ratio from 1:10 to 10:1, more preferably in a weight ratio from 1:5 to 5:1, e.g., from 1:3 to 3:1, or from 1:2 to 2:1, or 1:1.

3. The composition according to claim 1, wherein said composition comprises from 1 to 100 mg of B. longum novaBLG1 and from 1 to 100 mg of B. psychaerophilum Q5, both in lyophilized form, preferably from 5 mg to 50 mg of B. longum novaBLG1 and from 5 mg to 50 mg of B. psychaerophilum Q5, even more preferably from 10 mg to 40 mg of B. longum novaBLG1, e.g., 20 mg or 30 mg of B. longum novaBLG1, and from 10 mg to 40 mg of B. psychaerophilum Q5, e.g., 20 mg or 30 mg of B. psychaerophilum Q5.

4. The composition according to claim 1, wherein said composition comprises from 5 to 20 mg of B. longum novaBLG1 and from 5 to 20 mg of B. psychaerophilum Q5, both in lyophilized form, preferably 10 mg of B. longum novaBLG1 and 10 mg of B. psychaerophilum Q5.

5. The composition according to claim 1, wherein said composition is administered in daily doses ranging from 1.0×10⁶ CFU/day to 1.0×10¹² CFU/day, preferably from 1.0×10⁸ CFU/day to 1.0×10¹⁰ CFU/day, e.g., about 1.0×10⁹ CFU/day.

6. The composition according to claim 1, wherein said composition is for use as a medicament, preferably for use in the treatment of male fertility.

7. The composition for use according to claim 6, wherein said composition is for use in a method of treatment, preferably in a method of preventive or curative treatment, of male urinary diseases, preferably of benign prostatic hyperplasia (BPH).

8. A strain of bacteria selected from the group comprising or alternatively consisting of:
Bifidobacterium psychraerophilum “Q5,” deposit number DSM 33131; or
Bifidobacterium longum subsp. longum “novaBLG1,” deposit number DSM 34338;
and mixtures thereof.

9. The strain of bacteria according to claim 8, wherein said strain of bacteria is for use as a medicament, preferably for use in the treatment of male fertility.

10. The strain of bacteria for use according to claim 9, wherein said strain of bacteria is for use in a method of treatment, preferably in a method of preventive or curative treatment, of male urinary diseases, preferably of benign prostatic hyperplasia (BPH).

11. A method comprising:
administering a composition of claim 1 to a subject that has a male urinary disease.

12. The method of claim 11, wherein the subject has benign prostatic hyperplasia (BPH).

13. The method of claim 11, wherein the composition comprises Bifidobacterium psychraerophilum “Q5” DSM 33131 and Bifidobacterium longum subsp. longum “novaBLG1” DSM 34338, in a weight ratio from 1:10 to 10:1, more preferably in a weight ratio from 1:5 to 5:1, e.g., from 1:3 to 3:1, or from 1:2 to 2:1, or 1:1.

14. The method of claim 11, wherein the composition comprises from 1 to 100 mg of B. longum novaBLG1 and from 1 to 100 mg of B. psychaerophilum Q5, both in lyophilized form, preferably from 5 mg to 50 mg of B. longum novaBLG1 and from 5 mg to 50 mg of B. psychaerophilum Q5, even more preferably from 10 mg to 40 mg of B. longum novaBLG1, e.g., 20 mg or 30 mg of B. longum novaBLG1, and from 10 mg to 40 mg of B. psychaerophilum Q5, e.g., 20 mg or 30 mg of B. psychaerophilum Q5.

15. The method of claim 11, wherein the composition comprises from 5 to 20 mg of B. longum novaBLG1 and from 5 to 20 mg of B. psychaerophilum Q5, both in lyophilized form, preferably 10 mg of B. longum novaBLG1 and 10 mg of B. psychaerophilum Q5.

16. The method of claim 11, wherein the composition is administered in daily doses ranging from 1.0×10⁶ CFU/day to 1.0×10¹² CFU/day, preferably from 1.0×10⁸ CFU/day to 1.0×10¹⁰ CFU/day, e.g., about 1.0×10⁹ CFU/day.
